(19) United States
(12) Patent Application Publication
(10) Pub. No.: US 2019/0133442 A1
(43) Pub. Date: May 9, 2019

(54) INTRAOCULAR PRESSURE SENSOR

(71) Applicant: California Institute of Technology, Pasadena, CA (US)

(72) Inventors: Vinayak Narasimhan, Pasadena, CA (US); Radwanul H. Siddique, Pasadena, CA (US); Jeong Oen Lee, Pasadena, CA (US); Hyuck Choo, San Marino, CA (US)

(21) Appl. No.: 16/122,732

(22) Filed: Sep. 5, 2018

Related U.S. Application Data
(60) Provisional application No. 62/554,648, filed on Sep. 6, 2017.

(51) Int. Cl. A61B 3/16

(52) U.S. Cl. CPC A61B 3/16; A61B 2562/12; A61B 2562/0285

(57) ABSTRACT

By adopting nature's biopolymer-phase-separation process, a highly scalable biomimetic bottom-up nanofabrication method is developed to create low-aspect-ratio bioinspired nanostructures (BINS) on freestanding silicon-nitride (Si3N4) membranes. Unlike previous high-aspect-ratio nonstructures that focused on replicating optical antireflection and bactericidal properties, the IOP sensor with BINS (or BINS-IOP sensor) of the present disclosure has a pseudo-periodic arrangement and dimensions that control short-range scattering to enhance omnidirectional optical transmission and angle independence while also exhibiting anti-biofouling properties of high-aspect-ratio nanostructures, which typically rely on physical cell lysis. In some embodiments, the BINS-IOP sensor can have a low-aspect-ratio, which displays strong hydrophilicity to form an aqueous anti-adhesion barrier for proteins and cellular fouling without cell lysis.

### CROSS-REFERENCE TO RELATED APPLICATIONS

This application claims priority to and benefit of U.S. Provisional Application No. 62/554,648 entitled “Longtail Glasswing Butterfly Inspired Biofouling-resistant Biophotonic Nanostructures for Implants”, filed Sep. 6, 2017, the disclosure of which is incorporated herein by reference in its entirety for all purposes.

### STATEMENT REGARDING FEDERALLY-SPONSORED RESEARCH OR DEVELOPMENT

This invention was made with government support under Grant No. EY024582 awarded by the National Institutes of Health. The government has certain rights in the invention.

### FIELD

Various aspects of the disclosure relate to an intraocular pressure sensor.

### BACKGROUND

Glaucoma is a leading cause of blindness, affecting an estimated four million Americans and seventy million individuals globally. As glaucoma typically affects the elderly, the aging demographic trends indicate that this disease will continue to be an ever-increasing socioeconomic burden to society. Elevated intraocular pressure (“IOP”) is a major risk factor for glaucoma, and IOP monitoring is the single most important clinical management tool. Today, an estimated 8-10% of Americans and 5-6% of people in other developed nations depend on implantable medical devices to support or rebuild organs and other functions of the body during their lifetime. Consequently, there is an urgent need to develop medical implant technologies that are biocompatible (e.g., anti-biofouling or biofouling-resistant) while maintaining high performance and reliability.

### SUMMARY

Disclosed are embodiments of IOP sensors and methods for fabrication of the same. In some embodiments, the IOP sensor can include: a first substrate having a recess on a first surface; and a second substrate disposed on the first surface of the first substrate such that the recess of the first surface and the second substrate form a cavity. The second substrate has a plurality of structures on a surface that opposes the cavity.

The plurality of structures can have an aspect ratio between 0.15 to 0.90. In some embodiments, the plurality of structures has an aspect ratio of approximately 0.45. The plurality of structures can have varying sizes and shapes. The plurality of structures can be a plurality of nanostructures that have an average inter-structural period can have a range between 300-500 nanometers. In some embodiments, the average inter-structural period is 100-200 nanometers. The plurality of structures can have a circular or oval shape.

The first substrate of the IOP sensor can be made of silicon (Si). The second substrate of the IOP sensor can be made of silicon nitride (Si₃N₄). The IOP sensor can further include a third substrate disposed on a portion of the second surface of the second substrate; and a fourth substrate disposed on the third substrate. The third substrate can be made of silicon dioxide (SiO₂) and the first and fourth substrates can be made of silicon (Si).

The cavity of the IOP sensor can further include two or more trenches that are perpendicular to a length of the cavity. The trenches are located on a surface of the cavity that opposes the second substrate.

Also disclosed is a method for fabricating an intraocular pressure sensor. The method can include: spin-coating a first substrate assembly with a solution of polymers; evaporating a portion of the solution of polymers to form a plurality of islands on the first substrate assembly; removing the plurality of islands to form a first mask on the first substrate assembly, the first mask having a plurality of openings after removal of the islands; depositing a layer of metal-oxide on the first mask; removing the layer of metal-oxide to form a plurality of structures have an average aspect ratio of approximately 0.45 on the first substrate; and placing the first substrate assembly on a second substrate having a slot to form an optical cavity. The plurality of structures of the first substrate is placed over the slot of the second substrate.

The solution of polymers can include a first and a second polymer. The first polymer can be hydrophobic, and the second polymer can be hydrophilic or less hydrophobic. In some embodiment, the solution of polymers can be a solvent of methyl ethyl ketone with a solvent mass ratio of 35%. The first polymer can be polystyrene, and the second polymer can be poly-methyl-methacrylate.

Spin-coating the first substrate assembly can include: accelerating a spin of the first substrate from rest to 3500 rotation per minute (RPM) in 1.5 seconds; and spinning the first substrate assembly at 3500 RPM for 30 seconds. While spin-coating the first substrate assembly, the coating chamber can be maintained at a relative humidity between 40 to 50 percent.

Removing the islands from the first substrate can include: rinsing the first substrate assembly in cyclohexane between 1 to 3 minutes; and drying the first substrate assembly in a stream of nitrogen. Next, a layer of metal-oxide is deposited on the first mask. The layer of metal-oxide can be a 30 nm thick layer of Al₂O₃. Finally, the first substrate assembly is hermetically sealed to the second substrate to create an IOP sensor.

The islands, openings, and structures can generally be of any desired size. In many embodiments described herein, the plurality of islands is a plurality of nano-islands, the plurality of openings are a plurality of nano-openings, and the plurality of structures is a plurality of nanostructures. Those of ordinary skill in the art understand the term “nano” to imply a broad range of sizes that, as a matter of convenience, are typically expressed on the nanometer scale. The embodiments described herein can be practiced with nano-islands, nano-openings, and nanostructures having a largest length or width dimension from 0.1 nanometers to 10,000 nanometers, or 1 nanometer to 1000 nanometers, to name a few. The embodiments described herein can be practiced at dimensions less than 0.1 nanometers and more than 10,000 nanometers as well. Other example embodiments are provided herein.

Also disclosed is a second method for fabricating an intraocular pressure sensor. The second method can include: providing a first substrate layer having a plurality of structures; and disposing the first substrate layer on a second substrate having a slot to form an optical cavity. The plurality of structures can have an inter-structural period of 450 nm and an aspect ratio of 0.45. In the final IOP assembly, the plurality of structures of the first substrate layer is placed directly over the slot to form an optical cavity.

The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the disclosed subject matter.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIGS. 1-4 illustrate IOP sensors in accordance with some aspects of the disclosure.

FIGS. 5A-5E are process flow diagrams for fabricating a portion of IOP sensors shown in FIGS. 1-4 in accordance with some aspects of the disclosure.

FIG. 6A is a process flow chart for fabricating IOP sensors shown in FIGS. 1-4 in accordance with some aspects of the disclosure.

FIGS. 6B-6E illustrate a process for fabricating nanostructures in accordance with some aspects of the disclosure.

FIGS. 6F-6J illustrate a spin-coating process for fabricating nanostructures in accordance with some aspects of the disclosure.

FIG. 7 illustrates a process for fabricating the IOP sensor shown in FIG. 1.

FIGS. 8A-8E are process flow diagrams for fabricating a portion of IOP sensors shown in FIGS. 1-4 in accordance with some aspects of the disclosure.

FIG. 9A illustrates a near-field scatter profile of nanostructures of IOP sensors shown in FIGS. 1-4 in accordance with some aspect of the disclosure with period of 200 nm

FIG. 9B illustrates a near-field scatter profile of nanostructures having an average inter-structure period of 300 nm.

FIG. 10A illustrates an angle-transmittance profile of membrane with no nanostructures.

FIG. 10B illustrates a simulated angle-transmittance profile of membrane with no nanostructures.

FIG. 11A illustrates an angle-transmittance profile of nanostructures of IOP sensors shown in FIGS. 1-4 in accordance with some aspect of the disclosure.

FIG. 11B illustrates a simulated angle-transmittance profile of nanostructures of IOP sensors shown in FIGS. 1-4 in accordance with some aspect of the disclosure.

FIG. 12A is a bar graph illustrating the adhesion force of bovine serum albumin on various types of IOP sensors, including the IOP sensor shown in FIG. 1.

FIG. 12B is a bar graph illustrating the adhesion force of streptavidin on various types of IOP sensors, including the IOP sensor shown in FIG. 1.

FIG. 12C is a bar graph illustrating the adhesion force of E. coli bacteria on various types of IOP sensors, including the IOP sensor shown in FIG. 1.

FIG. 13A show fluorescent micrographs for various types of IOP sensors, including the IOP sensor shown in FIG. 1.

FIG. 13B is a bar graph illustrating cell density on various types of IOP sensors, including the IOP sensor shown in FIG. 1.

FIG. 14 is a bar graph illustrating cell mortality ratio on various types of IOP sensors, including the IOP sensor shown in FIG. 1.

FIG. 15 is a table listing various properties of synthetic and natural nanostructures, including the nanostructures used in the IOP sensor shown in FIG. 1.

FIGS. 16A and 16C illustrate the angle-dependent property (Fabry-Perot resonance) of a conventional IOP sensor at 90° and at less than 90°, respectively.

FIGS. 16B and 16D illustrate the angle-dependent property (Fabry-Perot resonance) of a IOP sensors shown in FIGS. 1-4 at 90° and at less than 90°, respectively.

FIG. 17A is a graph illustrating resonance shifts of the cavity of the IOP sensor shown in FIG. 1.

FIG. 17B illustrates a test configuration of the angle-dependency test.

FIG. 17C is a graph illustrating the peak shift in the reflected resonance spectra as a function of incidence angles of conventional and the IOP sensor shown in FIG. 1.

FIG. 17D is a graph illustrating the intensity profile as a function of incidence angles of conventional and the IOP sensor shown in FIG. 1.

FIG. 17E is a graph illustrating the pressure error profile as a function of incidence angles of conventional and the IOP sensor shown in FIG. 1.

FIG. 17F is a graph illustrating the performance of the IOP sensor shown in FIG. 1 at normal incidence.

FIG. 18A is a graph illustrating spectra intensity collected from continual IOP measurements taken over 60-second intervals with an integration time of 10 milliseconds per spectrum.

FIGS. 18B and 18C are histograms illustrating the numbers of spectra at specific AA relative to the mean wavelength for the conventional sensor and the IOP sensor shown in FIG. 1, respectively.

FIG. 18D is a graph illustrating in vivo IOP error of a tonometry reading and various types of IOP sensors, including the IOP sensor shown in FIG. 1.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

### Overview

As previously noted, there is a need to develop medical implant technologies that are anti-biofouling or biofouling-resistant while maintaining high performance and reliability. Such multifunctional surfaces are often seen in nature, which boasts a plethora of nanostructures with a wide array of desirable properties. For example, recent work has revealed the multifunctionality of high-aspect-ratio biophotonic nanostructures found on the wings of insects such as glasswing butterflies, danger cicada, and dragonflies. These nanostructures are needle-like and vertically tapered. They exhibit remarkable multifunctionality including omnidirectional antireflection, self-cleaning, antifouling, and bactericidal properties. Such properties may prove to be useful for engineering biofouling-resistant optical medical devices. In nature, these nanostructures are postulated to be self-assembled through a phase separation of biopolymers in the amphiphilic phospholipid bilayer of wing-scale cells, followed by chitin deposition in the extracellular space.

The IOP sensor disclosed herein was inspired from multifunctional biophotonic nanostructures found on the transparent wings of the longtail glasswing butterfly (chorinea faunus), which can help advance the versatility of implantable IOP sensors. To develop the nanostructures for the IOP sensor, the surface and optical properties of the short-range-ordered nanostructures found on the wings of the longtail glasswing butterfly (hereinafter referred to as C. faunus) are characterized in detail. Research of the C. faunus' wings reveals that the C. faunus relies on relatively moderate-aspect-ratio chitin nanostructures to produce transparency that is a unique combination of anti-reflection and Mie scattering, which has not been observed in other transparent wings found in nature. By adopting nature's biopolymer-phase-separation process, a highly scalable biomimetic bottom-up nanofabrication method is developed to create low-aspect-ratio bioinspired nanostructures (BINS) on freestanding silicon-nitride (Si3N4) membranes. Unlike previous high-aspect-ratio nonstructures that focused on replicating optical antireflection and bactericidal properties, the IOP sensor with BINS (or BINS-IOP sensor) of the present disclosure has a pseudo-periodic arrangement and dimensions that control short-range scattering to enhance omnidirectional optical transmission and angle independence while also exhibiting anti-biofouling properties of high-aspect-ratio nanostructures, which typically rely on physical cell lysis. In some embodiments, the BINS-IOP sensor can have a low-aspect-ratio, which displays strong hydrophilicity to form an aqueous anti-adhesion barrier for proteins and cellular fouling without cell lysis. Empirical data collected on the low-aspect-ratio BINS-IOP sensor of the present disclosure show significant improvement in the sensor's optical readout angle, pressure-sensing performance, and biocompatibility during a one-month in vivo study.

### Bio-Inspired Nanostructures IOP Sensor

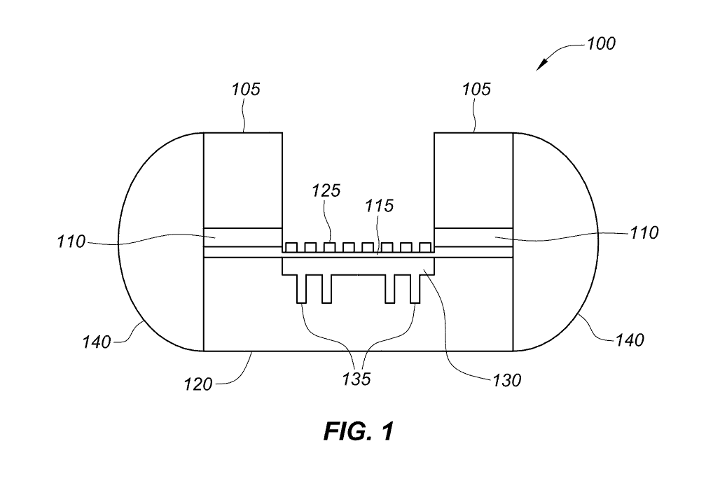

FIG. 1 illustrates a cross-section of an IOP sensor 100 in accordance with some embodiments of the present disclosure. IOP sensor 100 includes a first substrate layer 105, a second substrate layer 110, a third substrate layer 115, and a fourth substrate layer 120. First substrate layer 105 and fourth substrate layer 120 can be made of silicon. However, substrate layers 105 and 120 can be made of other types of semiconductor materials (e.g., germanium, gallium arsenide). First substrate layer 105 can have a thickness in range of 200-400 μm (micrometer). In some embodiments, first substrate layer 105 can have a thickness of 300 μm. Second substrate layer 110 can be an insulating layer, which can be made of silicon dioxide or other suitable insulating substrates. Second substrate layer 110 can have a thickness in a range of 1-4 μm. In some embodiments, second substrate layer 110 can have a thickness of 2 μm.

Third substrate layer 115 can be made of silicon-nitride (Si3N4) or other thermodynamically stable and/or biocompatible compounds. Third substrate layer 115 includes a plurality of nanostructures 125 on one of the surfaces that opposes fourth substrate layer 120, which includes a cavity 130. Third substrate layer 115 can have a thickness range between 200-400 nm (nanometer). In some embodiments, third substrate layer 115 can have a thickness of 300 nm. Nanostructures 125 can have an average aspect ratio in a range of 0.15 to 0.90. In some embodiments, nanostructures 125 can have an average aspect ratio of 0.45 and can be made of the same material as third substrate layer 115. Alternatively, nanostructures 125 and third substrate layer 115 can be made of different materials. Nanostructures 125 can have a circular, oval (e.g., ellipsoidal), pyramidal, or cylindrical shape. In some embodiments, nanostructures 125 have an ellipsoidal shape. Nanostructures 125 can have an average inter-structural period (the center-to-center distance between two adjacent nanostructures) in a range of 300 to 500 nm. In some embodiments, nanostructures 125 can have an average inter-structural period of 100-200 nm.

Similar to first substrate layer 105, fourth substrate layer 120 can have a thickness range between 200-400 nm (nanometer). In some embodiments, fourth substrate layer 120 can have a thickness of 300 nm.

Cavity 130 of fourth substrate layer 120 can also include one or more trenches 135 that are orthogonal to the length of cavity 130. In some embodiments, cavity 130 can have four trenches 135, which can function as reservoirs to wick and store any leftover (and unwanted) liquid that may be in cavity 130. For example, during a process to hermetically seal cavity 130 and third substrate layer 115, trenches 135 can collect and store any sealant or epoxy that may have overflow or leak into cavity 130. In some embodiments, cavity 130 can have a depth in a range of 3-10 μm. In one embodiment, cavity 130 can have a depth of 4 μm.

IOP sensor 100 can also include a sealing layer 140 that hermetically seals substrate layers 105, 110, 115, and 120 together to form a Fabry-Perot cavity. Sealing layer 140 can be made of a medical grade epoxy or other types of sealant materials. Sealing layer 140 can encompasses the entire circumference of substrate layers 105 and 120.

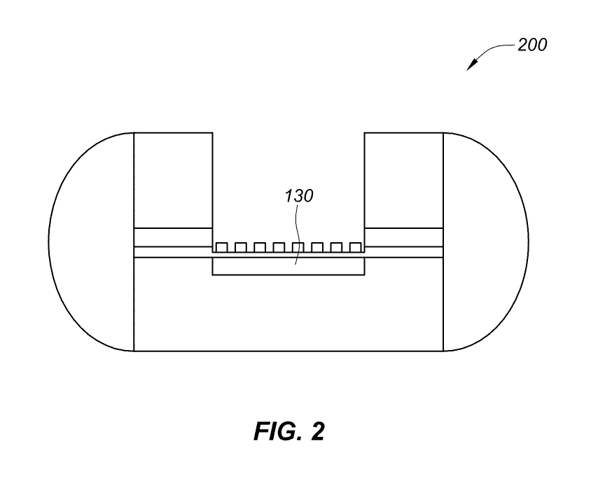

FIG. 2 illustrates a cross-section of an IOP sensor 200 in accordance with some embodiments of the present disclosure. IOP sensor 200 includes all of the features of IOP sensor 100 as described above except for the absent of trenches 135 (see FIG. 1) in cavity 130.

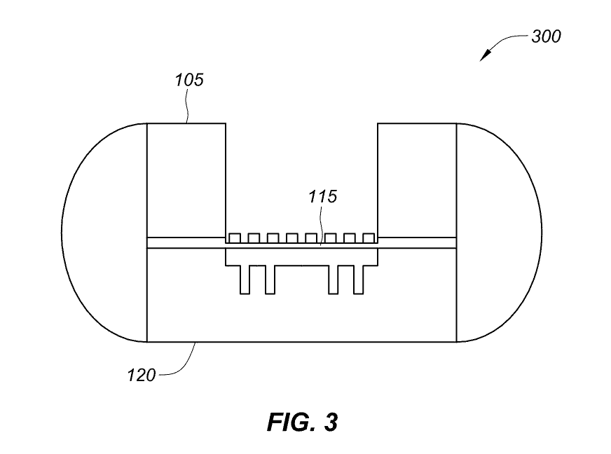

FIG. 3 illustrates a cross-section of an IOP sensor 300 in accordance with some embodiments of the present disclosure. IOP sensor 300 includes all of the features of IOP sensor 100 as described above except for the absent of an insulating layer (i.e., substrate layer 110 of FIG. 1) between first substrate layer 105 and third substrate layer 115.

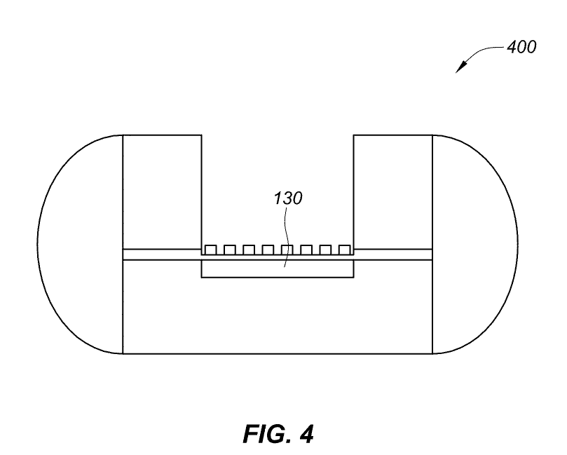

FIG. 4 illustrates a cross-section of an IOP sensor 400 in accordance with some embodiments of the present disclosure. IOP sensor 300 includes all of the features of IOP sensor 300 as described above except for the absent of trenches 135 (see FIG. 1) in cavity 130.

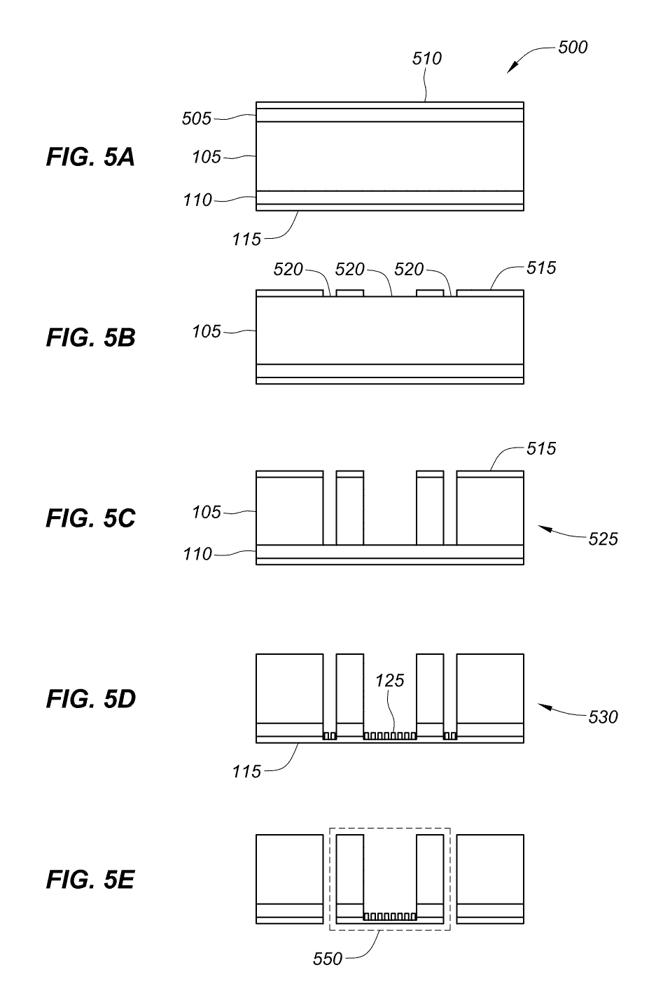

FIGS. 5A-5E illustrate a process 500 for fabricating a portion 550 of IOP sensor 100 in accordance with some embodiments of the present disclosure. These figures will be discussed concurrently. Portion 550 can be a top portion of IOP sensor 100 that includes first substrate layer 105, second substrate layer 110, and third substrate layer 115 having the plurality of nanostructures 125 (see FIG. 1). The fabrication of portion 550 starts at FIG. 5A where first substrate layer 105 is coated on both sides with layers of silicon dioxide (SiO2) using thermal oxidation. It should be noted that other type of coating process can be used in place of thermal oxidation. This coating process forms substrate layers 110 and 505, as shown in FIG. 5A. In some embodiments, substrate layers 110 and 505 are 2 μm thick. First substrate layer 105 can be a double-sided polished silicon wafer. Next, substrate layers 110 and 505 are coated with layers of silicon nitride (Si3N4) using low pressure chemical vapor deposition (LPCVD) or other coating methods. The LPCVD process can be used to form substrate layers 115 and 510. In some embodiments, substrate layers 115 and 510 are 400 nm thick.

In FIG. 5B, substrate layers 505 and 510 of FIG. 5A are removed using reactive ion etching (ME) and buffered oxide etch (BOE), respectively. Next, a layer of aluminum oxide (Al₂O₃) 515 is deposited onto substrate layer 105 using an electron beam evaporator or other coating methods. In some embodiments, substrate layer 515 has a thickness of 300 nm. Substrate layer 515 is then patterned using photolithography to form the precursor shapes for openings (e.g., slots) 520, which is finally formed through another buffered oxide etch process, which removed portions of the layer of aluminum oxide that was exposed during the photolithography process.

In FIG. 5C, the remaining substrate layer 515 can functions as a hard mask for the next etching process, which remove portions of layer 105 that are not protected by substrate layer 515, down to substrate layer 110. After portions of layer 105 are removed by the etching process, substrate assembly 525 is formed.

FIG. 5D illustrates substrate assembly 530 having nanostructures 125 already formed and exposed. Prior to forming nanostructures 125, substrate layer 515 is removed from substrate assembly 525 through BOE. Next, substrate layer 110 is removed using BOE to expose silicon-nitride layer 115.

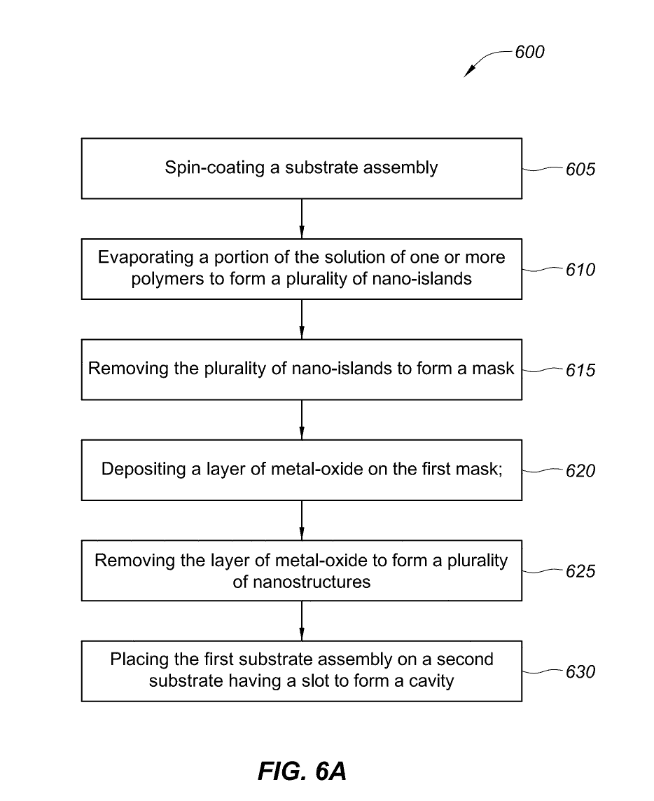

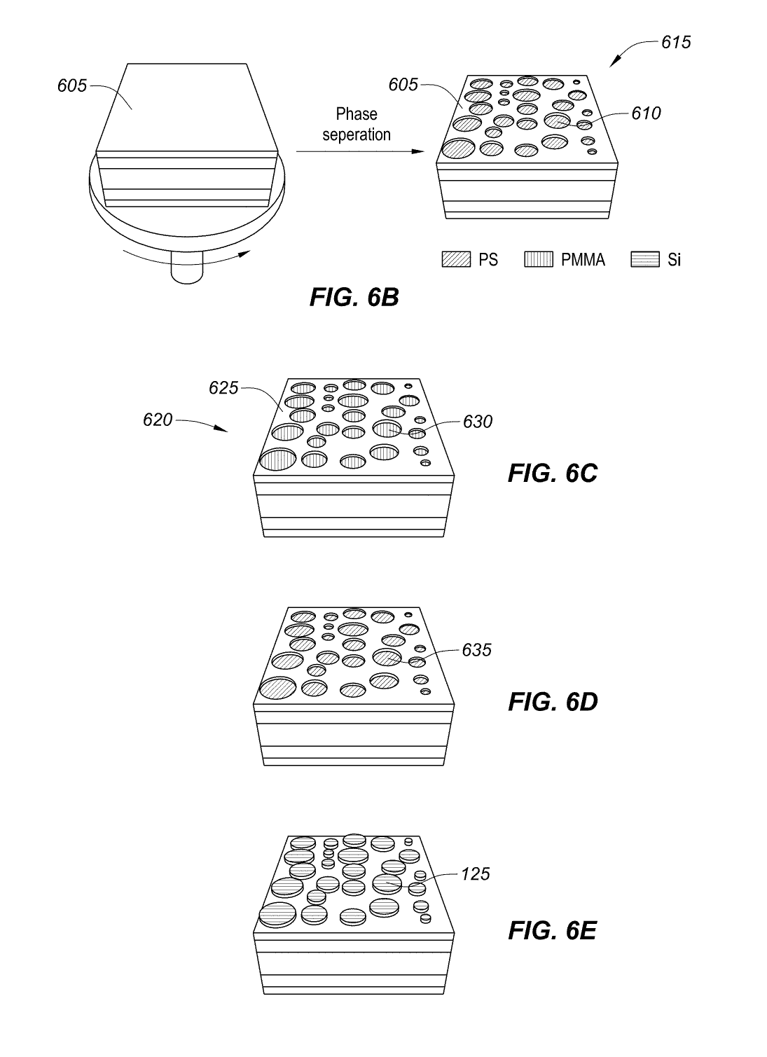

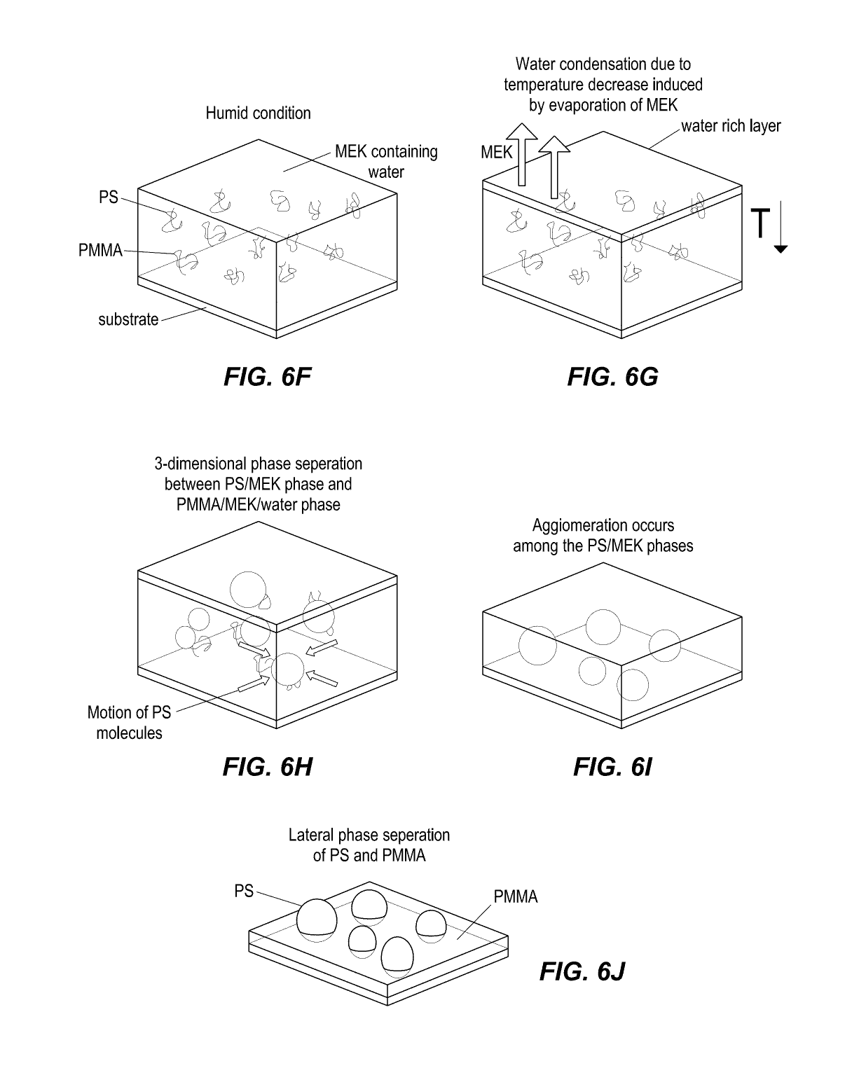

To form substrate assembly 530, a nanostructures-forming process 600 is performed. Referring to FIG. 6A, which illustrates nanostructures forming process 600 in accordance with some embodiments of the present disclosure. Process 600 starts at 605 where substrate assembly 525 without substrate layers 110 and 515 (see FIG. 5C) is spin-coated with a solution of two or more polymers. In some embodiments, the solution of two or more polymers can have two different polymers, a first and second polymer. The first polymer can be more hydrophobic than a second polymer, which can be hydrophilic. In some embodiments, the first and second polymers can be polystyrene (PS) and poly-methyl methacrylate (PMMA), respectively. The PS can have a molecular weight in a range of 15-25 kg/mol. In some embodiments, the PS can have a molecular weight of 19.1 kg/mol. The PMMA can have a molecular weight in a range of 5-15 kg/mol. In some embodiments, the PMMA can have a molecular weight of 9.59 kg/mol. The polymers can be dissolved in a solution of methyl ethyl ketone (MEK). In some embodiments, the solution of polymers can have a mass ratio of 65% (PMMA) and 35% (PS). The concentration of the solution can be kept at 15-25 mg/ml.

In some embodiments, for the spin-coating process, substrate assembly 525 (without substrate layers 110 and 515, hereinafter referred to as substrate assembly 525′) is accelerated at a rate of 2000 rpm/s for 1.5 seconds to reach 3500 rpm. Substrate assembly 525′ is then spin-coated with the solution of polymers while being rotated at 3500 rpm for 30 seconds. The relative humidity of the spin-coating process can be maintained between 40% and 50%. Due to the difference in relative solubilities of the PS and the PMMA in the MEK solution, de-mixing (e.g., separation) of the blended polymers occurs in the coating layer while substrate 525′ is being spin-coated.

FIGS. 6B-6J graphically illustrate nanostructures forming process 600. FIGS. 6A-6J will now be discussed concurrently. FIG. 6B illustrates subprocess 610 of FIG. 6A. At 610, a portion of the solution of polymers is evaporated after the spin-coating procedure. When substrate 525′ spins during the coating process, water condensation begins at humidity levels >35%, which causes a layer of water-rich solution to form at the air solution interface due to the difference in evaporation rate between water and MEK. The water then starts to condense from the air into the solution because of the evaporation of MEK (see FIG. 6G), which decreases the temperature on top below the dew point. Because of the high concentration of water, a 3-dimensional phase separation occurs between PS/MEK and PMMA/MEK/water (see FIG. 6H). This causes the PS molecules start to agglomerate (see FIG. 6I). With the further evaporation of MEK, the PS/MEK, and PMMA/MEK/water phases reach the same height. When film 605 is dried, a purely lateral morphology is formed and the PS molecules in the solution form ellipsoidal-shaped PS nano-islands 610 on the surface of film 605 (see FIG. 6F-6J). This forms substrate assembly 615.

FIG. 6C illustrates subprocess 615 of FIG. 6A where substrate assembly 615 (from FIG. 6B) is rinsed in cyclohexane and dried in a stream of nitrogen (N2) gas to remove PS islands 610. In some embodiments, substrate assembly 615 is rinsed in cyclohexane between 1-3 minutes. In one embodiment, substrate assembly 615 is rinsed in cyclohexane for 2 minutes. This forms substrate assembly 620 having PMMA layer 625 with nano-openings 630 (where the PS islands 610 used to be located). PMMA layer 625 then functions as a hard mask (e.g., template) for the next material coating process.

FIG. 6D illustrates subprocess 620 where a layer of aluminum oxide is deposited onto substrate assembly 620 with PMMA layer 625 functioning as a hard mask. In some embodiments, the layer of aluminum oxide is deposited onto substrate assembly 620 using electron beam evaporation.

FIG. 6E illustrates subprocess 625 where aluminum oxide islands 635 are removed using BOE to yield a layer of silicon nitrate with nanostructures 125. This process creates substrate assembly 530 as shown in FIG. 5D.

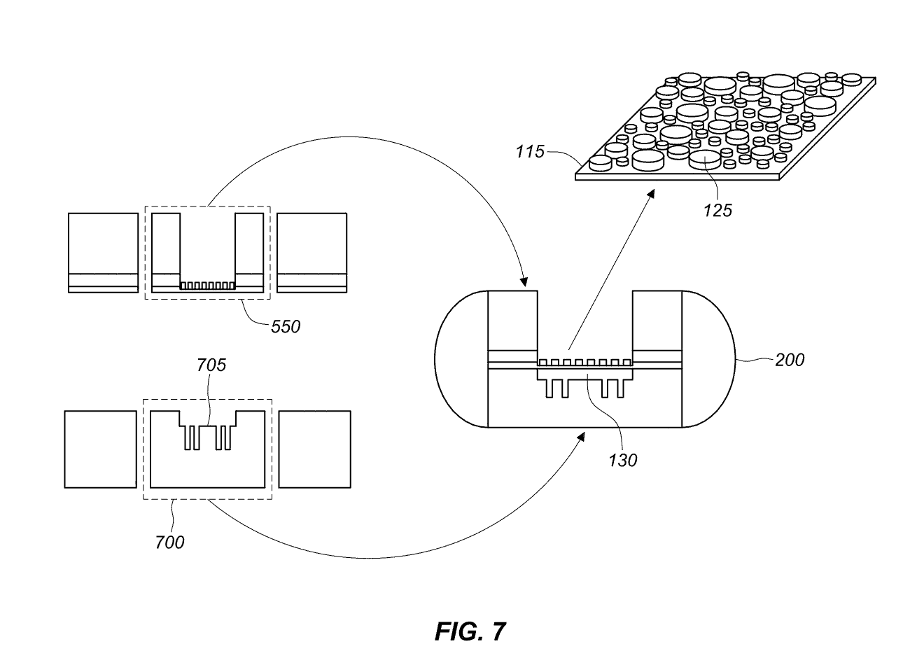

Referring now to FIG. 5E, portion 550 is removed from substrate assembly 530, which will be assembled with a bottom substrate 700 of FIG. 7 to form cavity 130 of IOP sensor 100. Referring both to FIGS. 6A and 7, at 630, a first or top substrate assembly (portion 550) is placed on second substrate 700 having a slot 705 to form cavity 130. In some embodiments, portion 550 and second substrate 700 are hermitically sealed using an epoxy 710 that encompasses the edges of portion 550 and substrate 700.

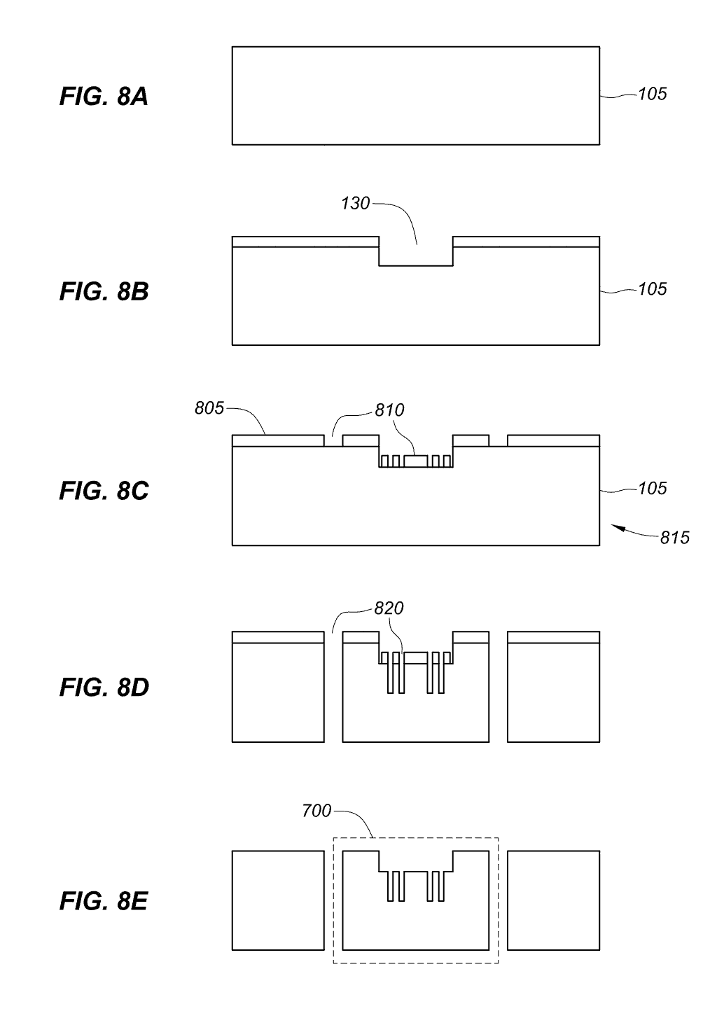

FIGS. 8A-8E illustrate a process 800 for fabricating second (bottom) substrate 700 in accordance with some embodiments of the present disclosure. Process 800 starts in FIG. 8A where a polished silicon wafer (substrate layer 105) is provided. In FIG. 8B, a photoresist mask is deposited onto substrate 105. Next, in some embodiments, cavity 130 is created using reactive ion etching. In FIG. 8C, after removing the photoresist layer, a layer of aluminum oxide (Al₂O₃) 805 is deposited onto substrate 105 using an electron beam evaporator. Layer 805 is then patterned with openings 810 on the top-most surface and also within cavity 130. This yields substrate assembly 815.

In FIG. 8D, substrate assembly 815 is etched to create trenches 820. In FIG. 8E, substrate layer 805 is removed using BOE and bottom substrate 700 is formed, which is then combined with portion 550 (see FIG. 7).

### Experimental Tests and Data

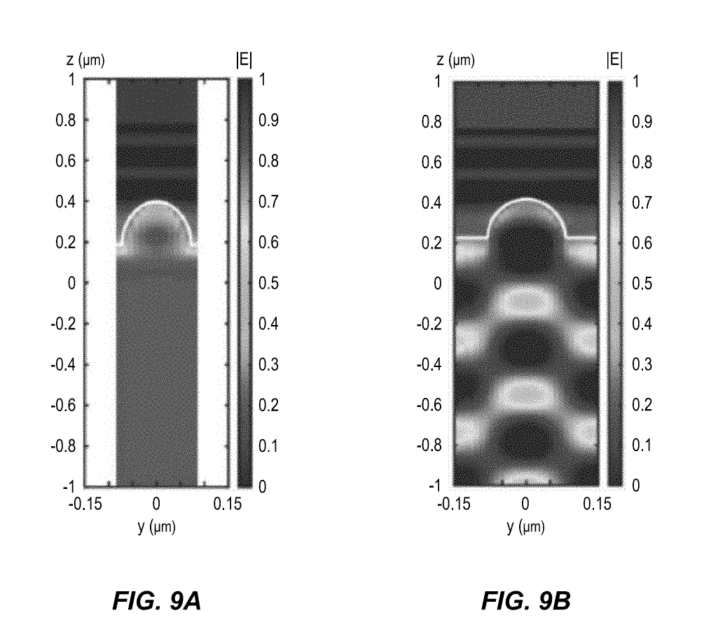

Empirical studies of the nanostructures of IOP sensor 100 show that the variation in average inter-structural periods plays an important role in the extent of light scattering. To confirm the scattering properties of IOP sensor 100, finite-difference time-domain (FDTD) simulations were performed on nanostructures with periods of 150 and 300 nm at 420-nm wavelength (FIGS. 2E-F). Although both groups have the same structural height and diameter, nanostructures with a 150 nm inter-structural period do not alter the transmitted field (see FIG. 9A). However, nanostructures with a 300-nm period scatter the transmitted light as shown in FIG. 9B. An additional advantage of nanostructures having an average inter-structural period of 150 nm is the wetting property. The static contact angles measured for the nanostructures with an average inter-structure period of 150 was 105°.

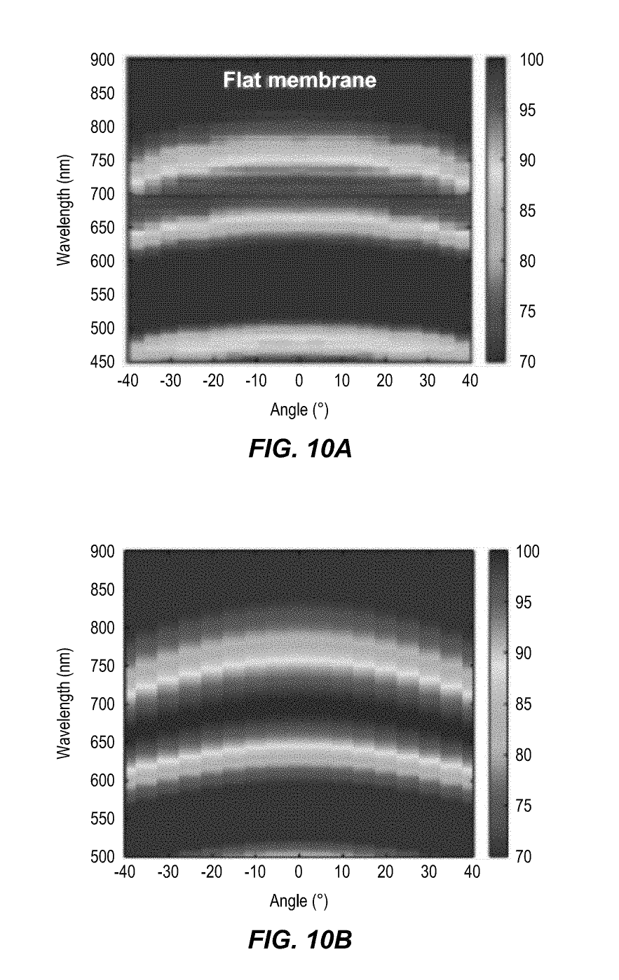

The optical properties of IOP sensor 100 was characterized using an angle resolved transmission spectroscopy in the visible near infrared (VIS-NIR) range. The results are then compared with the results of a conventional (flat) IOP sensor without the Si₃N₄ nanostructures. The conventional IOP sensor produced a transmission peak around 705 nm due to thin-film interference and its peak location blue-shifted 30 nm at 40° incident angle (see FIG. 10A). This agrees with the results from analytical thin-film modeling (see FIG. 10B), which shows relatively the same transmission peak and peak location.

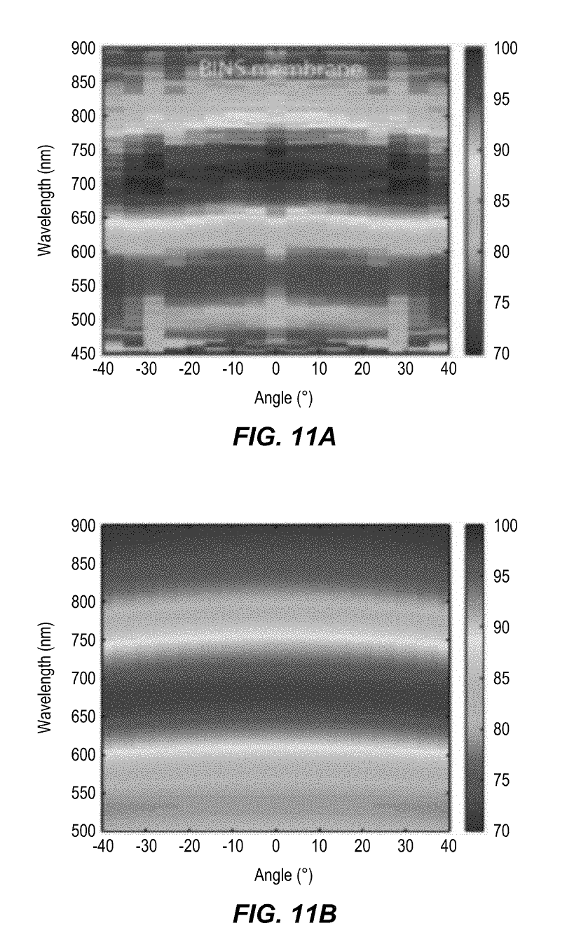

The integration of nanostructures on an IOP sensor (e.g., IOP sensor 100) broadens the transmission-peak profile and moves its center to 715 nm, but most noticeably it limits the magnitude of peak shift to 15 nm at 40°, indicating a significant reduction in angle dependence (see FIG. 11A), 3D simulation of the fabricated structures further confirms the improved angle-independent transmittance (see FIG. 11B). This angle-independent property occurs when the short-range-ordered nanostructures introduce optimally controlled isotropic scattering, which then broadens the reflection and transmission angles involved in the coherent process of thin-film interference.

In vitro adhesion tests of representative proteins, prokaryotes, and eukaryotes were performed on IOP sensor 100 and conventional IOP sensor (e.g., sensor without nanostructures or flat IOP sensor) with lysine-coated glass slides as positive controls. Experimental results show that conventional IOP sensor is moderately hydrophilic, which has a contact angle between 35-40° (the angle between a droplet's edge and the surface). Moderate hydrophilic surfaces are known to promote cell adhesion and proliferation due to increased adsorption of proteins as compared to high hydrophilic surfaces, which have a contact angle of less than 20°. The nanostructures of IOP sensor 100 is highly hydrophilic, which is achieved by adjusting the surface roughness and by varying the aspect-ratios of the nanostructures from 0.15 to 0.90. Empirical data shows that an aspect ratio of 0.45 provides an optimum balance of high hydrophilicity and anti-adhesion properties. Accordingly, in some embodiments, nanostructures of IOP sensor 100 have an aspect ratio of approximately 0.45. Due to IOP sensor 100 strong hydrophilicity (contact angle less than) 20°, an aqueous barrier forms on the surface and limits protein adsorption and cell adhesion to provide an overall anti-adhesion character to IOP sensor 100.

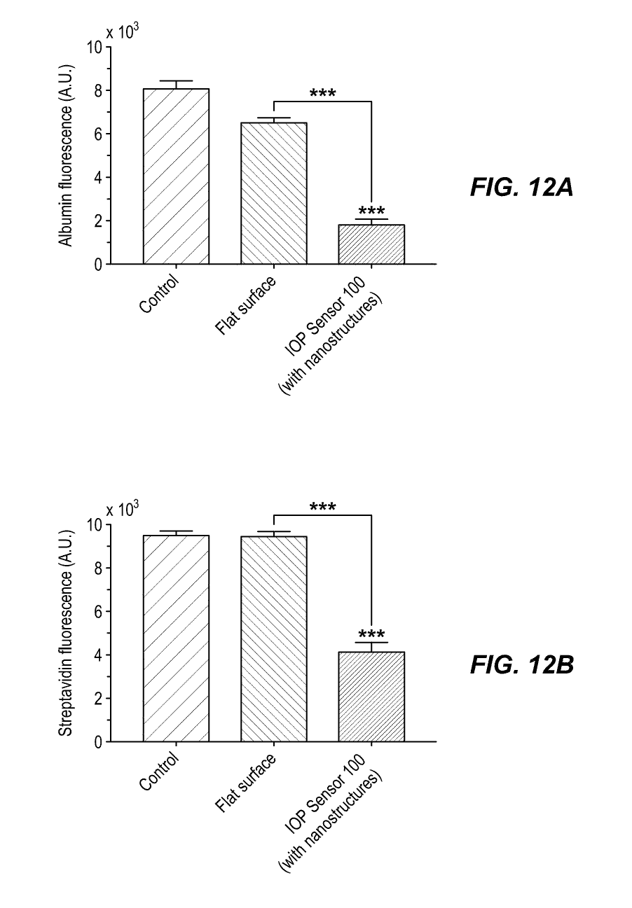

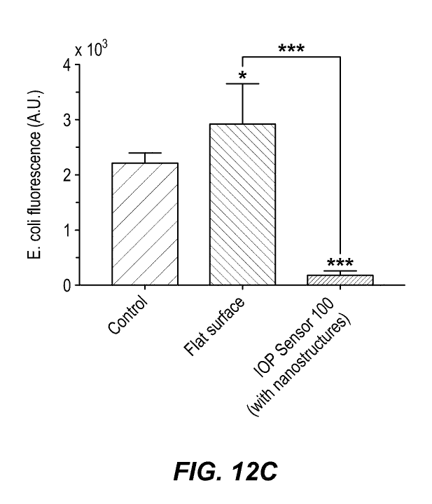

Surface adhesion tests of two representative proteins and bacteria were performed on a control IOP sensor, a flat IOP sensor, and IOP sensor 100. The representative proteins were: (1) fluorescent-labelled bovine serum albumin (BSA) for its cardinal role in blood-material interactions and high non-specific binding affinity to the surfaces of biomaterials; and (2) streptavidin for its specific binding affinity to Si3N4 surfaces. The bacteria used in the adhesion test were E. coli. FIGS. 12A and 12B show the adhesion tests results. FIG. 12A shows that the control IOP and conventional IOP sensors have 3 times the adhesion level than IOP sensor 100 for BSA. FIG. 12B shows that the control IOP and conventional IOP sensors have at least twice the adhesion level than IOP sensor 100 for streptavidin.

FIG. 12C shows that the adhesion level of E. coli bacteria for IOP sensor 100 is significantly less than both the control and conventional IOP sensors. Additionally, the SEM image of individual bacterial cells on IOP sensor 100 shows no disruption to their shape, indicating no physical lysis.

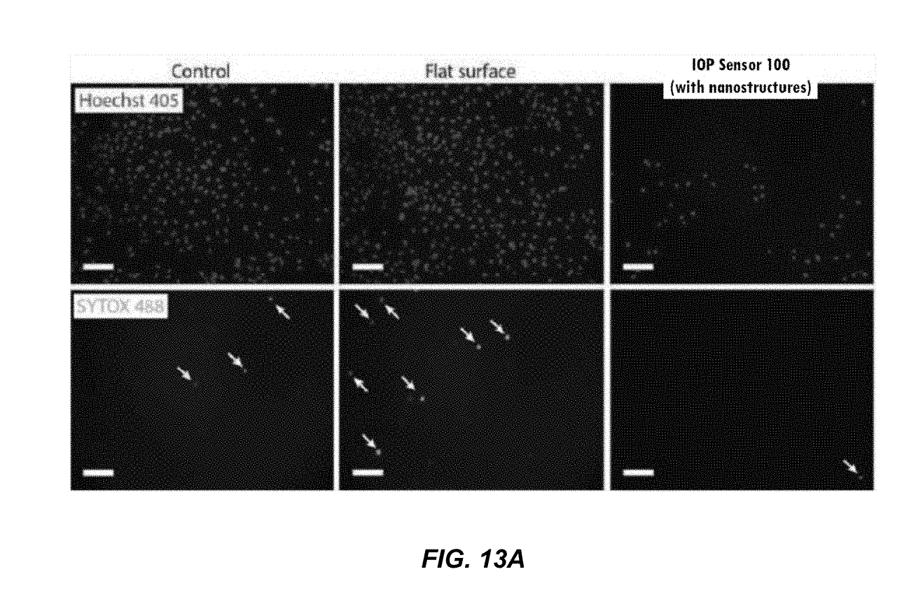

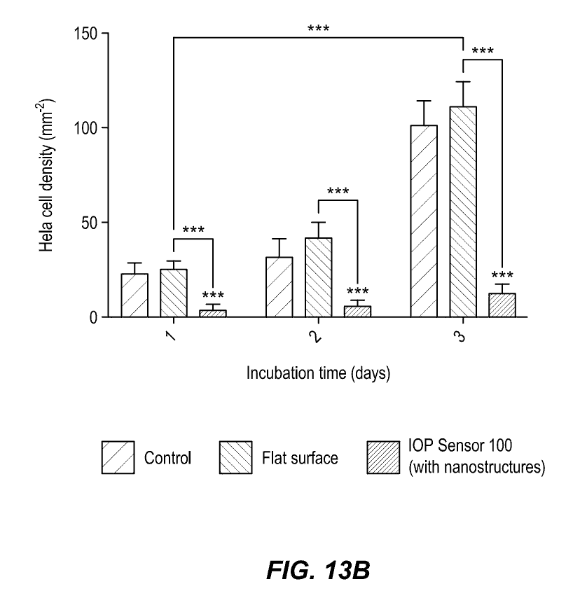

Further adhesion tests were performed using the HeLa cell line, which is a representative eukaryote having exceptional robustness, aggressive growth rate, and adherent nature. After 72 hours, the adherent cell density on the conventional IOP sensor was eight times greater than that on IOP sensor 100 (see FIGS. 13A and 13B).

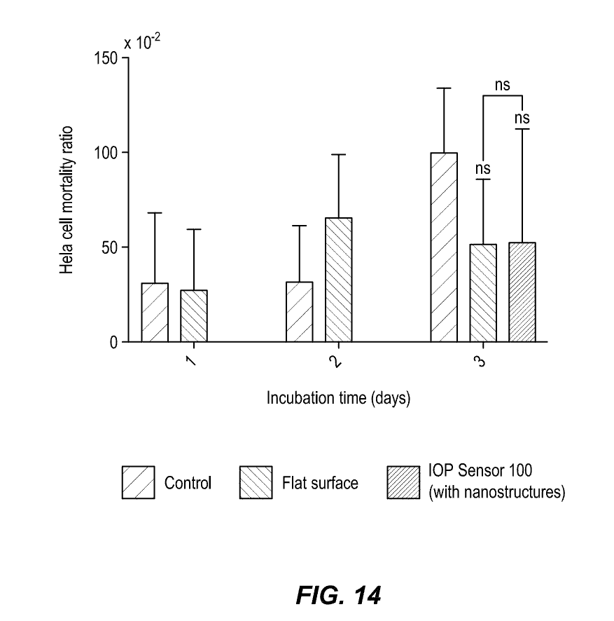

Mortality ratio tests were also performed on a control IOP sensor, conventional IOP sensor, and IOP sensor 100. The number of dead cells to the number of living cells, was computed for each surface of the sensors every 24 hours over a 72-hour period. The difference in the mortality ratios of the two surfaces after 72 hours was not statistically significant (FIG. 14), which suggested that the nanostructures on IOP sensor 100 inhibited eukaryote adhesion and proliferation without inducing cell death.

These results highlight the advantage of the anti-biofouling approach based on strong hydrophilicity and anti-adhesion properties. High or moderate aspect-ratio nanostructures either with tapered sharp tips or dome-shaped tips as implemented in IOP sensor 100 display potent geometry-dependent bactericidal properties that induce large stresses and deformation on cell walls regardless of their surface chemical composition and actively promote autogenous lysis when placed in contact with mammalian cells. Such anti-biofouling approach relying on physical lysis could undesirably damage tissues surrounding implants and elicit inflammation.

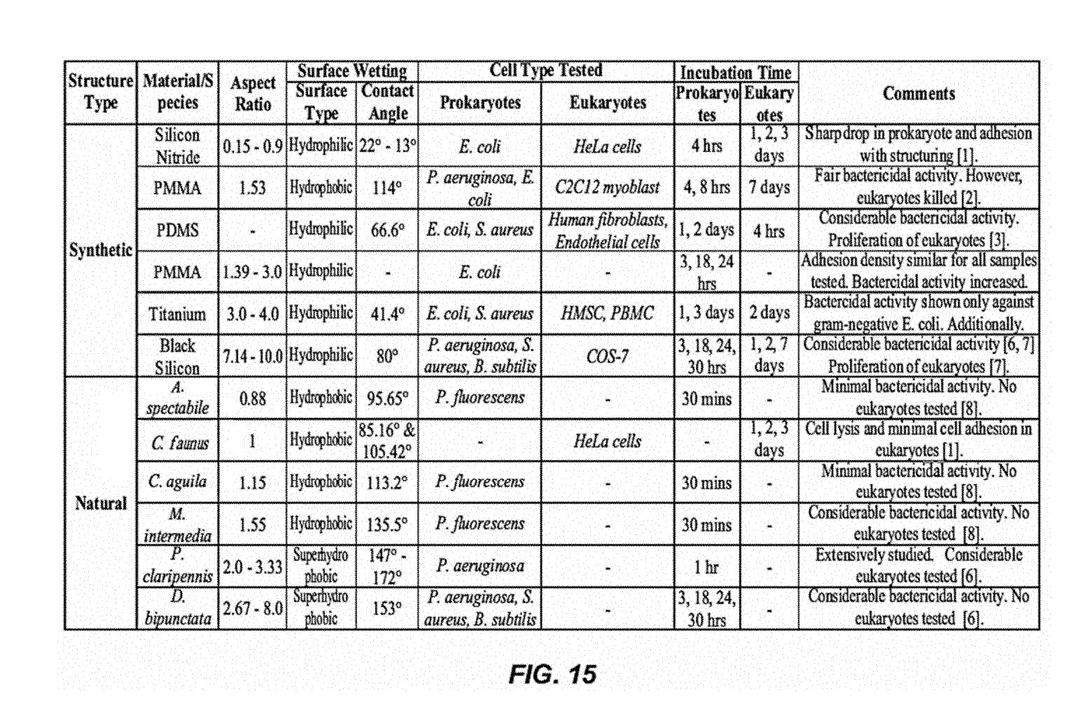

FIG. 15 shows physical lyses occur on either natural or synthetic nanostructured surfaces if the aspect-ratio of the nanostructures is 1 or greater. Hence, by keeping the aspect-ratio of IOP sensor 100 at approximately 0.45, the anti-adhesion property was leveraged to prevent biofouling without causing any physical lysis. Generally, hydrophilic materials are more resistant to bacterial adhesion than hydrophobic materials because hydrophobic microbes adhere more strongly to surfaces than hydrophilic ones. Additionally, the hydrophilicity of the nanostructures on IOP sensor 100 originates from surface topology, which can provide better long-term reliability over chemical-coating methods.

### BINS IOP Sensor Characterization

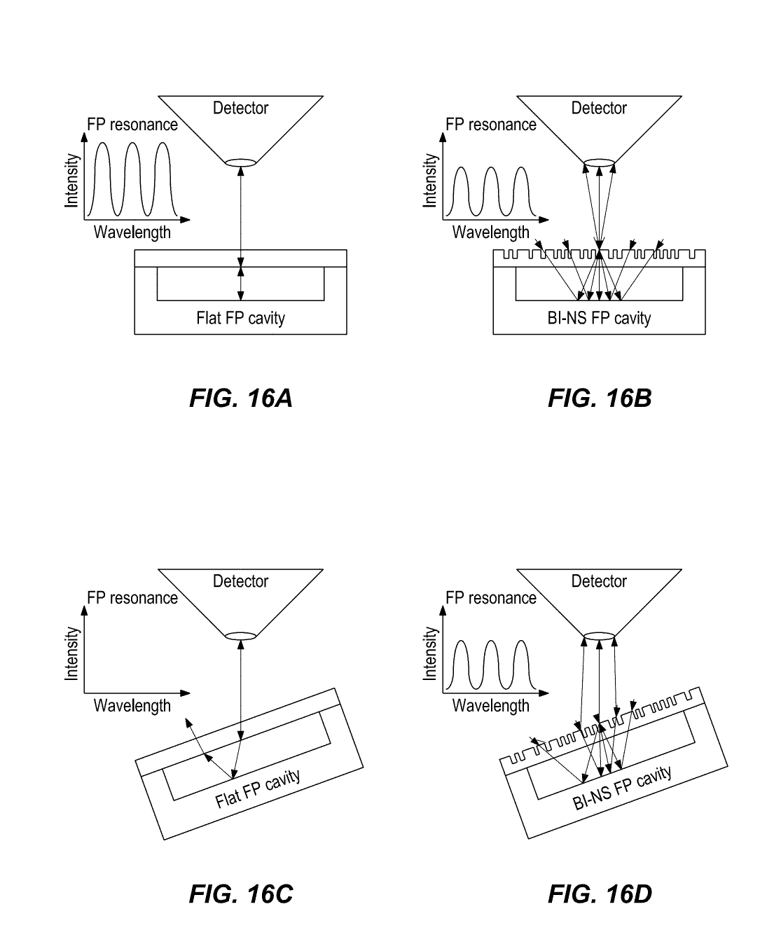

Conventional sensors with a flat-surfaced membrane have successfully provided in vivo IOP measurements, but its accuracy and usability suffered from narrow readout angles inherent to conventional FP-resonators (FIGS. 16A and 16C). As shown in FIG. 16A, under normal incidence, the signal-to-noise ratio (SNR) is high. However, at an angle beyond a certain threshold angle, the SNR falls to zero—illustrating the high angle dependency of conventional IOP sensor. Additionally, the lifespan of conventional IOP sensors was shortened by occasional but serious biofouling.

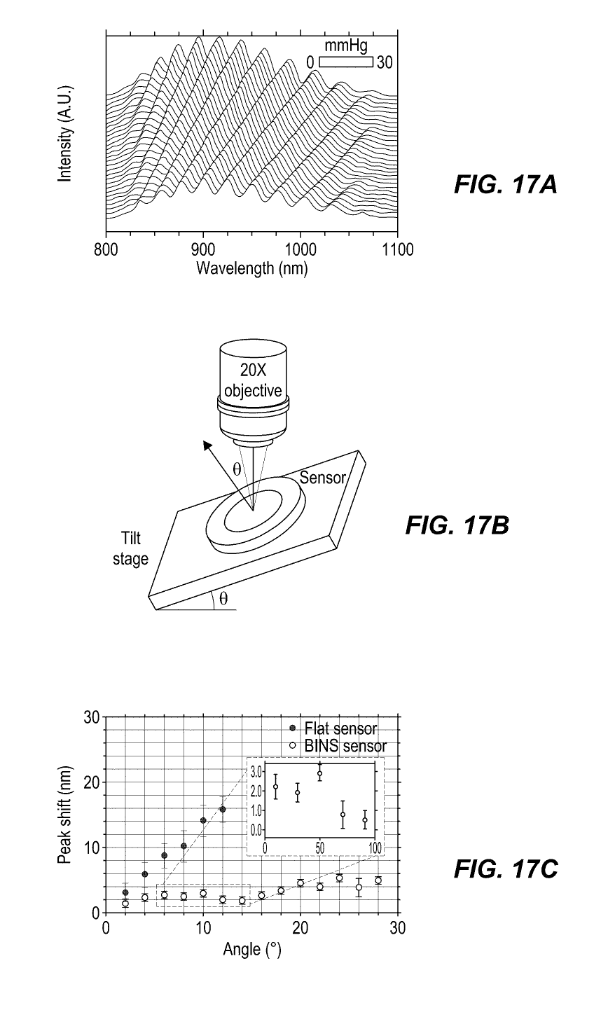

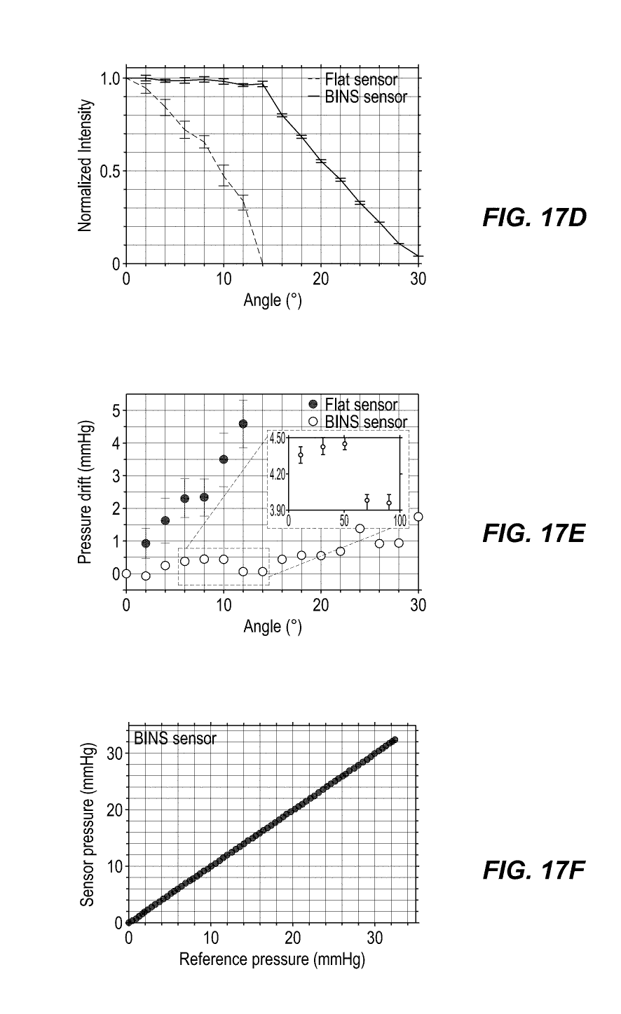

In contrast, the SNR of IOP sensor 100 remain relatively high at normal and at a wide range of angles (see FIGS. 16B and 16D). Further a study of angle dependency on readout angle of 0° was performed using an optical arrangement similar to the optical arrangement shown in FIGS. 16B, 16D, and 17B to compare measurements from IOP sensor 100 and a conventional IOP sensor at 1 atmosphere. Conventional IOP sensor produced a maximum resonance shift of 16 nm at incident angle of 12° (FIG. 17C). In contrast, IOP sensor 100 (BINS sensor) produced shifts of 2 nm at 12° and 5 nm at 30°. Decay in the intensity of reflected resonance was also measured as a function of the incident angle (FIG. 17D). For the flat-surfaced sensor, the intensity decayed to zero when the incident angle reached 12° while the signal from IOP sensor 100 remained detectable until 30°. The IOP-measurement error of the flat-surfaced sensor reached 4.59 mmHg at 12° (FIG. 17E), which is approximately 46% of the physiological IOP range observed in humans (10-20 mmHg) and exceeds the clinically-accepted error range of ±2 mm Hg. On the other hand, the IOP-measurement error of IOP sensor 100 was 0.07 and 1.74 mmHg at 12° and 30°, respectively. These results highlight the exceptional wide-angle performance of IOP sensor 100 (e.g., BINS-integrated sensor).

Finally, when tested in a pressure-controlled chamber interfaced with a digital pressure gauge, IOP sensor 100 showed excellent linearity (correlation factor: ˜1.00) over the clinically interested range from 0 to 30 mmHg (FIG. 17F). The maximum readout error was 0.26 mmHg, approximately four times lower than that of the conventional sensor (1 mmHg).

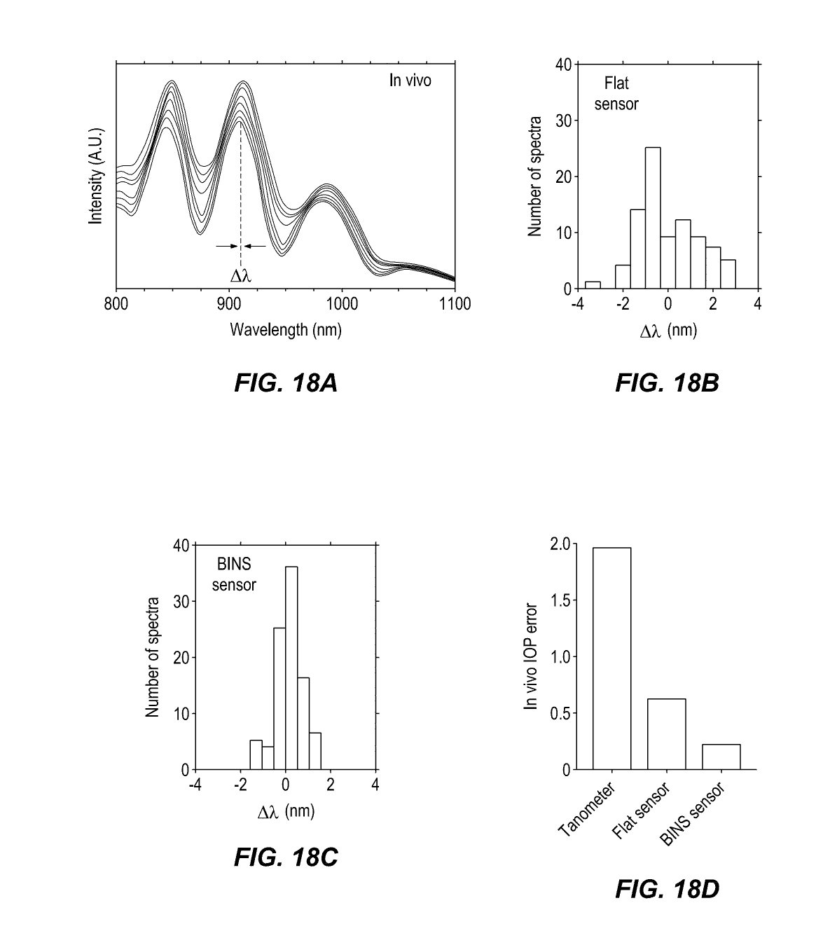

Both IOP sensors 100 and conventional sensors were implanted individually inside the anterior chambers of two New Zealand white rabbits to investigate in vivo optical performance and biocompatibility. One hundred spectra with highest signal-to-noise ratio (SNR) were averaged in 1 minute of continual measurement to produce a single IOP readout. To examine the stability of sensor measurements, the shift Δλ of the most prominent peak in each spectrum of the set was then computed with respect to the mean (FIG. 18A). The standard deviation (SD) of Δλ of IOP sensor 100 was 0.6 nm as opposed to 1.3 nm observed for the conventional (flat-surfaced) sensor (FIGS. 18B and 18C). Additionally, the SD of IOP measurements produced using IOP sensor 100 was 0.23 mmHg as opposed to 0.64 and 1.97 mmHg calculated from measurements concurrently obtained using the conventional sensor and tonometry, respectively (FIG. 18D). The angle independence enhanced by IOP sensor 100 improved the stability and accuracy of the optical measurements against potential error sources such as respiratory movements, subtle eye motions, and detector misalignment.

Both sensors were retrieved after one month of implantation to quantify the surface cell growth and to assess biocompatibility. Confocal fluorescence microscopy was used to determine the extent of tissue growth and cellular viability at the time of retrieval. DAPI was used to localize all constituent cells while phalloidin, which selectively binds to actin, was used as an indicator of cellular processes and health. Additionally, matrix metalloproteinases-2 (MMP-2) was used as an indicator of inflammation for its role in various inflammatory and repair processes.

Z-stacked multi-channel immunofluorescence images of the conventional IOP sensor and IOP sensor 100 were generated (not shown). Based on the data collected from these images, 59% of the conventional was covered by tissue, and there was a healthy tissue growth at the time of extraction. Additionally, MMP-2 was observed over the membrane of the conventional sensor, which could have triggered the extensive cell migration towards this region.

In comparison, approximately 5% of the surface of IOP sensor 100 was covered by tissue, which was a 12-fold improvement over the conventional sensor, and there was no detectable MMP-2 signal. This suggests that the cell signaling and migration patterns present on the flat-surfaced (e.g., conventional) sensor were absent on the BINS-integrated sensor (e.g., IOP sensors 100, 200, 300, and 400). This also indicates no inflammation occurred post-implantation and highlights the promising role of the BINS towards significantly improving in vivo biocompatibility of medical implants.

The foregoing description of the embodiments has been presented for the purposes of illustration and description. It is not intended to be exhaustive or to limit the inventive subject matter to the precise form disclosed. Many modifications and variations are possible in light of the above teaching. It is intended that the scope of the present inventive subject matter be limited not by this detailed description, but rather by the claims of this application. As will be understood by those familiar with the art, the present inventive subject matter may be embodied in other specific forms without departing from the spirit or essential characteristics thereof.

Where a discrete value or range of values is set forth, it is noted that that value or range of values may be claimed more broadly than as a discrete number or range of numbers, unless indicated otherwise. For example, each value or range of values provided herein may be claimed as an approximation and this paragraph serves as antecedent basis and written support for the introduction of claims, at any time, that recite each such value or range of values as “approximately” that value, “approximately” that range of values, “about” that value, and/or “about” that range of values. Conversely, if a value or range of values is stated as an approximation or generalization, e.g., approximately X or about X, then that value or range of values can be claimed discretely without using such a broadening term. Those of skill in the art will readily understand the scope of those terms of approximation. Alternatively, each value set forth herein may be claimed as that value plus or minus 5%, and each lower limit of a range of values provided herein may be claimed as the lower limit of that range minus 5%, and each upper limit of a range of values provided herein may be claimed as the upper limit of that range plus 5%, and this paragraph serves as antecedent basis and written support for the introduction of claims, at any time, that recite those percentile variations.

Reference in the specification to “one embodiment” or “an embodiment” means that a particular feature, structure, or characteristic described in connection with the embodiment is included in at least one embodiment of the inventive subject matter. The appearances of the phrase “in one embodiment” in various places in the specification are not necessarily all referring to the same embodiment.

In many instances entities are described herein as being coupled to other entities. It should be understood that the terms “coupled” and “connected” (or any of their forms) are used interchangeably herein and, in both cases, are generic to the direct coupling of two entities (without any non-negligible intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

Additionally, as used herein and in the appended claims, the singular forms “a,” “an,” and “the” include plural referents unless the context clearly dictates otherwise.

1. An intraocular pressure sensor comprising:
a first substrate having a recess on a first surface; and
a second substrate on the first surface of the first substrate such that the recess of the first surface and the second substrate form a cavity, the second substrate having a plurality of structures on a second surface that opposes the cavity.

2. The intraocular pressure sensor of claim 1, wherein each of the plurality of structures has an aspect ratio (height/width) between 0.15 to 0.90.

3. The intraocular pressure sensor of claim 1, wherein each of the plurality of structures has an aspect ratio (height/width) of approximately 0.45.

4. The intraocular pressure sensor of claim 1, wherein the plurality of structures is a plurality of nanostructures that has an average inter-structural period in a range between 300-500 nanometers.

5. The intraocular pressure sensor of claim 1, wherein the plurality of structures is a plurality of nanostructures that has an average inter-structural period of 450 nanometers.

6. The intraocular pressure sensor of claim 1, wherein the second substrate comprises silicon nitride (Si₃N₄).

7. The intraocular pressure sensor of claim 1, wherein the first substrate comprises silicon.

8. The intraocular pressure sensor of claim 1, further comprising:
a third substrate disposed on a portion of the second surface of the second substrate; and
a fourth substrate disposed on the third substrate.

9. The intraocular pressure sensor of claim 8, wherein the third substrate comprises silicon dioxide (SiO₂) and the first and fourth substrates comprise silicon (Si).

10. The intraocular pressure sensor of claim 1, wherein the cavity includes two or more trenches that are perpendicular to a length of the cavity, wherein the trenches are located on a surface of the cavity that is opposing the second substrate.

11. A method for fabricating an intraocular pressure sensor, the method comprising:
spin-coating a first substrate assembly with a solution of polymers;
evaporating a portion of the solution of polymers to form a plurality of islands on the first substrate assembly;
removing the plurality of islands to form a first mask on the first substrate assembly, the first mask having a plurality of openings after removal of the islands;
depositing a layer of metal-oxide on the first mask;
removing the layer of metal-oxide to form a plurality of structures having an average aspect ratio of approximately 0.45 on the first substrate; and
placing the first substrate assembly on a second substrate having a slot to form a cavity, wherein the plurality of structures is placed over the slot.

12. The method of claim 11, wherein the solution of polymers comprises a first and a second polymer, wherein the first polymer is hydrophobic and the second polymer is hydrophilic.

13. The method of claim 12, wherein the solution of polymers comprises a solvent of methyl ethyl ketone, wherein the two polymers have a mass ratio of 35% and 65%, respectively.

14. The method of claim 11, wherein the solution of polymers comprises a first and a second polymer, wherein the first polymer comprises polystyrene and the second polymer comprises poly-methyl-methacrylate.

15. The method of claim 11, wherein spin-coating the first substrate assembly comprises:
accelerating a spin of the first substrate from rest to 3500 rotation per minute (RPM) in 1.5 seconds; and
spinning the first substrate assembly at 3500 RPM for 30 seconds.

16. The method of claim 11, wherein spin-coating the first substrate assembly comprises maintaining a relative humidity between 40 to 50 percent.

17. The method of claim 11, wherein removing the islands comprises:
rinsing the first substrate assembly in cyclohexane between 1 to 3 minutes; and
drying the first substrate assembly in a stream of nitrogen.

18. The method of claim 11, wherein depositing the layer of metal-oxide on the first mask comprises depositing a 30 nm thick layer of Al₂O₃ onto the first mask.

19. The method of claim 11, further comprising hermetically sealing the first substrate assembly onto the second substrate.

20. The method of claim 11, wherein the plurality of islands is a plurality of nano-islands, wherein the plurality of openings is a plurality of nano-openings, and wherein the plurality of structures is a plurality of nanostructures.

21. A method for fabricating an intraocular pressure sensor, the method comprising:
providing a first substrate layer having a plurality of structures, wherein the structures have an aspect ratio of approximately 0.45; and
placing the first substrate layer on a second substrate having a slot to form an optical cavity, wherein the plurality of structures is placed over the slot.
